(19) United States
(12) Patent Application Publication
(10) Pub. No.: US 2026/0232325 A1
(43) Pub. Date: Aug. 13, 2026

(54) WINDLASS ROD FOR HEMOSTASIS SYSTEMS

(71) Applicant: Flavio Limontini, San Pietro Mosezzo (IT)

(72) Inventor: Flavio Limontini, San Pietro Mosezzo (IT)

(21) Appl. No.: 19/052,546

(22) Filed: Feb. 13, 2025

(51) Int. Cl. A61B 17/132; A61B 90/00

(52) U.S. Cl. CPC A61B 17/1327; A61B 90/08; A61B 2090/0811

(57) ABSTRACT

The patent describes a windless rod for a hemostasis system, wherein the hemostasis system incorporates a pressure band and at least one tourniquet, the ends of which are affixed to pressure band. Windless rod features a cavity into which is inserted the at least one tourniquet of the hemostasis system. Cavity of windless rod includes an opening allowing the insertion of the at least one tourniquet into cavity and its removal from the same.

### FIELD OF THE INVENTION

This invention concerns a windlass rod for a hemostasis system that can be used in first aid conditions to stop hemorrhages in major extremity.

### PRIOR ART

Systems of this type are already known in the art field, for example those described in European patent applications EP1753344 and EP2425786. This known system essentially incorporates a pressure band, at least one tourniquet affixed at its ends to said pressure band and at least one cylindrical windlass rod that extends along a longitudinal axis and that is equipped with a closed cavity into which the tourniquet is inserted. An example of the windless rod in this system is shown in FIGS. 5 and 6. Although the system is intuitive and accompanied by written instructions, the use of a system of this type requires personnel to undergo prior training. The general practice is therefore to prepare packages containing training systems as distinct from packages containing systems intended for actual use. According to conventions adopted to date, packages containing systems for training demonstrations include a blue color windlass bar, while single-use packages containing systems intended for actual emergency use include a black color windlass rod.

At the time of production, at least one tourniquet is inserted in the closed cavity of the windlass rod and then fastened to the pressure band. It follows that the manufacturer must prepare packages in which the windlass rod is blue in color, and packages in which the windlass rod is black. This makes it necessary to arrange two different types of packages and to differentiate production according to market demand.

It should however be considered that these hemostasis system packages have an expiry date, after which the package must be discarded even if the hemostasis system intended for actual use has never been used. Moreover, it may occur that the windlass rod included in a hemostasis system package has manufacturing defects or breakages of the plastic construction material, thus preventing its effective use.

### SUMMARY OF THE INVENTION

In view of these matters, one aim of this invention is to provide a windlass rod for a hemostasis system that can be reused for training purposes if the hemostasis system has reached its expiry date.

Another aim of this invention is to provide a windlass rod for a hemostasis system that can replace the original windlass rod in the package in cases where the original rod presents manufacturing defects, damage, and/or breakages.

These and other aims are achieved by this invention, which concerns a windlass rod having a cavity for insertion of at least one tourniquet into the cavity into the cavity. Interior walls of the cavity may be smooth. Alternatively, the interior cavity wall may be gnarled as a friction means for retaining at least one tourniquet in the cavity. The gnarled or roughened wall portion may be selected from a group comprising notched, scored, grooved or rifling, for additional friction on the at least one tourniquet therein.

A windless rod for a hemostasis system is used in a system that includes a pressure band and at least one tourniquet, wherein the tourniquet ends are attached to the pressure band.

The windlass rod, which has an elongated shape and extends along a longitudinal axis, features a cavity which the at least one tourniquet is inserted. The cavity in the windlass rod according to the innovation includes an opening allowing the insertion of at least one tourniquet into the cavity and its removal from said cavity.

This makes it possible to use a system according to the invention as required by contingent circumstances. For example, if the system is supplied with a windlass rod in an initial predetermined color (e.g. black), in which at least one tourniquet has been previously inserted into the respective cavity for actual use in a first aid situation, it is possible to remove the windlass rod of the first predetermined color from the at least one tourniquet and use a different windlass rod in a second predetermined color (e.g. blue), inserting the at least one tourniquet into the respective cavity through the opening in the same. This makes it possible to reuse, for example, a hemostasis system that has passed its expiry date to train the personnel who will be using an operational system of the same type within its expiry date for first aid applications, thus avoiding the need to discard the expired hemostasis system.

This makes it possible to supply a hemostasis system to stop major extremity hemorrhages, including at least two windlass rods of different colors in the package, wherein each windlass rod has a cavity that incorporates an opening to allow the insertion and removal of the at least one tourniquet of a system according to the invention.

As such, it is possible to simplify and decrease the cost of production of these hemostasis systems because each package can be used for actual first aid and or training.

If the first of the two windlass rods in the package is defective or broken such as to prevent it from being used, the second windlass rod can also be used for first aid operations, regardless of its color.

The opening in windlass rod cavity can be composed of a slot located on an angled axis with respect to the longitudinal axis of the windlass rod. This makes it possible to easily insert the at least one tourniquet into the cavity and to remove it from said cavity. In a form of implementation of this invention, the windlass rod can have a central portion of essentially oval or elliptical cross section in correspondence with the cavity. The cavity extends along a longitudinal axis parallel to the longitudinal axis of the windlass rod and offset with respect to the center of the central part with an oval or elliptical cross section. This imparts elevated robustness to the windlass rod when applying the necessary torque to the at least one tourniquet.

In one embodiment, the windlass rod is made of plastic and includes at least one flexible tab, located over the cavity, capable of being raised slightly to facilitate the insertion of the at least one tourniquet into the cavity, simultaneously protecting the slot that provides access to the internal cavity.

The windlass rod can include indicators, for example located in proximity to the slot that provides access to the cavity, helping to understand when the tourniquet(s) has/have been fed correctly into the cavity.

### BRIEF DESCRIPTION OF THE DRAWINGS

Further characteristics and advantages of this invention will be clarified by the following description, which is provided by way of illustration and without limitation with reference to the attached non-scale schematic drawings, wherein:

FIG. 1 shows a plan view of an exemplified and simplified implementation of a hemostasis system comprising a windlass rod according to this invention;

FIG. 2 is an enlarged side view of a windlass rod of the system shown in FIG. 1;

FIG. 3 is a sectional view, enlarged further, of plane III-III of the windlass rod depicted in FIG. 2;

FIG. 4 is a plan view of the windlass rod depicted in FIG. 2;

FIG. 4A is a section view of a gnarled inner wall of the windlass, comprising notched A, scored B, grooved C, or rifling D;

FIG. 5 is a side of a windlass rod fashioned in accordance with prior art;

FIG. 6 is an enlarged sectional view of plane V-V of the prior art windlass rod depicted in FIG. 5;

FIG. 7 is an elevation view of the windlass rod with cavity opening and parallel striated grooves on the rod exterior surface;

FIG. 8 is a top view of the windlass rod and proximity of the cavity openings;

FIG. 9 is a side view of the windlass rod and grooves on the rod external surface;

FIG. 10 is a perspective view of the windless rod with at least one tourniquet.

### DETAILED DESCRIPTION OF THE INVENTION

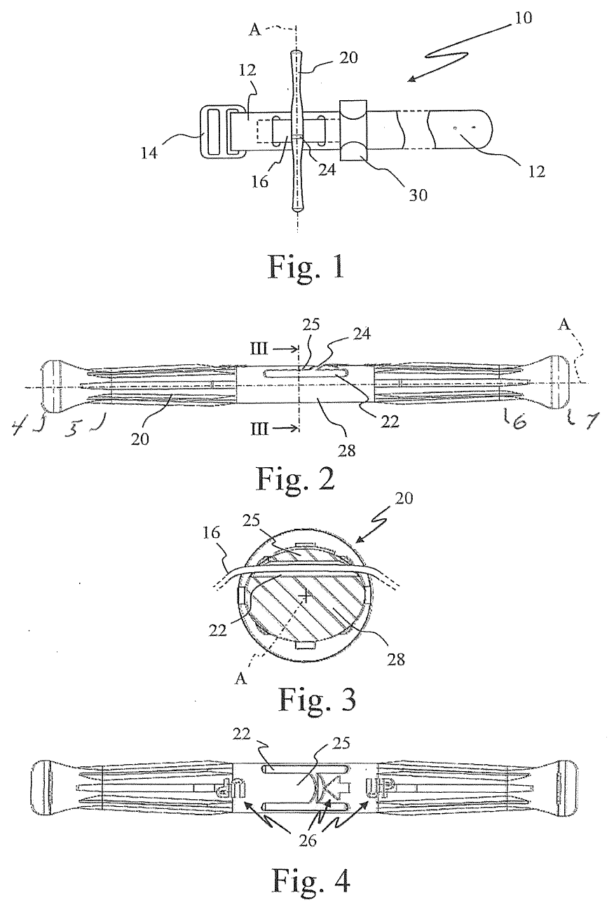

FIG. 1 is a schematic view of a hemostasis system 10 that includes a pressure band 12 to which is affixed, at one end, a sliding buckle 14. The other end, or the unrestrained end, of pressure band 12 is intended to be engaged in sliding buckle 14 after pressure band 12 has been wrapped around the injured limb in which a hemorrhage must be stopped. System 10 also incorporates a tourniquet 16 the ends of which are affixed to pressure band 12.

The at least one tourniquet 16 is inserted into a cavity 22, FIGS. 2 and 3, of a windlass rod 20 made of a molded plastic piece, having an elongate contoured cylindrical shape and extending along a longitudinal center axis A. The first end 3 of windlass rod 20 is round 4. A narrow contour 5 extends to a wider robust center portion 28, whereupon the rod extends to a second narrow contour 6 and a round second end 7. These contours make the windlass rod 20 distinctive and easier to locate in the medical bag and the wide central portion 28 makes the device easier to grasp. When the windlass rod central portion 28 is held in the palm, the cavity is apparent facilitating insertion of the at least one tourniquet 16. Cavity 22 of windlass rod 20 incorporates an opening 24 allowing the insertion of the tourniquet 16 into cavity 22. In the implementation described herein a single tourniquet 16 is depicted for the sake of clarity, but it should be noted that tourniquets 16 can be, for example, two in number, both affixed at their ends to opposite parts of pressure band 12, in such a way as to form two loops to insert into cavity 22.

System 10 further incorporates at least one locking device 30 affixed to pressure band 12 to hold windlass rod 20 in position and allow its release from the retention position. The locking device 30 can be composed, for example, a “C” shaped clamp. Following the application of pressure band 12 to the injured limb, windlass rod 20 can be rotated to tension the at least one tourniquet 16 and tighten it against pressure band 12. Once the compression applied is sufficient to stop the hemorrhage, windlass rod 20 can be held in position by locking device 30. The windlass rod 20 must however be released periodically from locking device 30 to reduce the compression exerted by the at least one tourniquet 16 on pressure band 12 applied to the injured limb and allowing a minimum blood flow to the limb. The windlass rod 20 will then be rotated again and returned to its position in the locking device 30 to stop the hemorrhage again.

As shown in FIG. 2, opening 24 that provides access to cavity 22 is composed of a slot that is peripherally remote and angled with respect to longitudinal center axis A of windless rod 20. Cavity 22 is located in a central bar portion 28 and it extends on a longitudinal axis parallel to longitudinal center axis A of windless rod 20 from the central rod axis A, toward the windlass rod exterior surface. Tab 25 positioned above cavity 22 is flexible and it can be raised slightly to facilitate insertion of the at least one tourniquet 16, simultaneously protecting the slot that forms access opening 24.

The view in FIG. 3 highlights the central portion 28 of windless rod 20, which has an essentially oval or elliptical shape in the area of the cavity 22 having a smooth interior wall 23. The cross section of central portion 28 can thus withstand the necessary force to perform its action on tourniquet(s) 16.

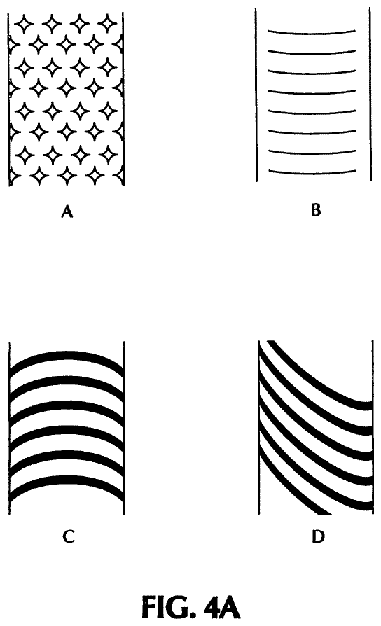

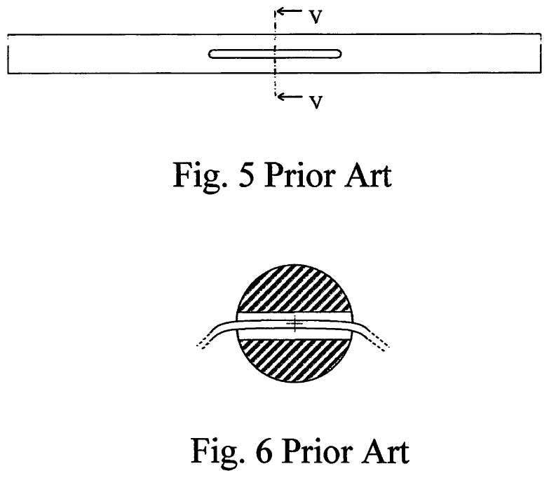

As shown in FIG. 4, indicators 26 may be marked or embossed on windless rod 20, helping to understand when the tourniquet(s) 16 has/have been fed correctly into cavity 22. Referring now to FIG. 4A, an interior cavity wall 23 and cavity opening 24 of the windlass rod may be gnarled as a friction means for retaining the at least one tourniquet 16 in cavity 22. The gnarled or roughened inner cavity wall portion 23 comprises a plurality of projections and depressions that provide friction on the pressure band in a configuration selected from a group comprising notched A, scored B, grooved C, rifled D, to provide additional friction on the pressure band and the at least one tourniquet 16 therein. Dimensions of these prominences or projections and crevices, of 1 to 3 mm, or more preferably, within a range of 1.5 to 1.75 mm, measured from the surrounding flat or planar interior cavity wall 23, are minimized for ease in threading and removal of at least one tourniquet 16 into the cavity 22. However, the gnarled/roughened inner cavity wall 23 becomes an effective friction means, particularly at the bridge piece 35, which lies over the cavity 22, when the tourniquet sliding buckle 14 is fastened on a hemorrhaging extremity and pressure is applied by rotation of the windlass rod.

In emergencies, first responders may drop smaller instruments such as the windlass rod adding to the stress and pressure of the situation. As such parallel grooves 33 in striated form are positioned on the bar outer surface as seen in FIG. 7. The longitudinal grooves enhance tactility of the windlass rod; i.e., the grooves cause the rod to be more tangible and increase capability of being felt or touched, thereby reducing a tendency to drop or otherwise displace the rod.

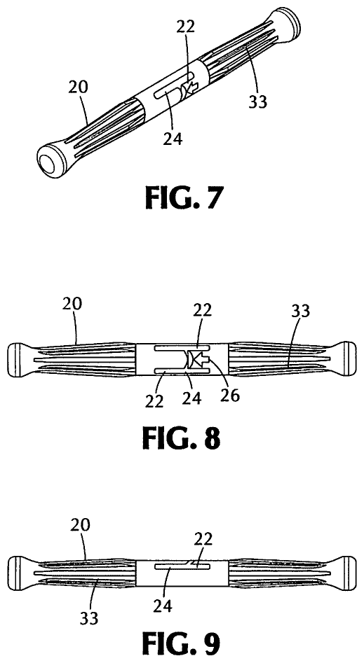

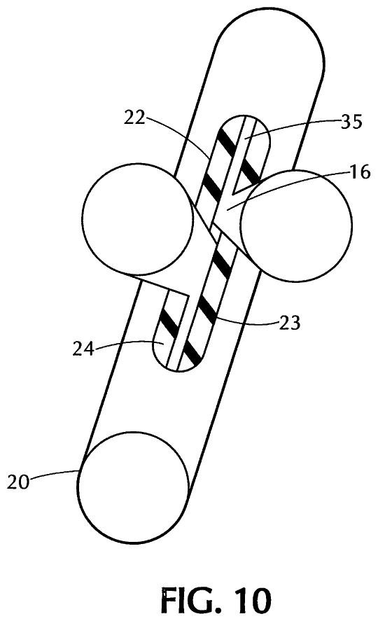

FIG. 8 is a top view of the windlass bar 20, wherein proximity of the cavity openings 24 facilitates insertion and removal of at least one tourniquet(s). In reference to FIG. 9 there is shown a side view of the windlass rod 20 and grooves 33 on external surface of the rod, the grooves 33 are parallel to axis A and to each other. FIG. 10 demonstrates the at least one tourniquet operative with the windlass rod.

In a hemostasis system 10 the windlass rod 20 is sealed within a package that contains at least two windlass rods 20 of different colors, to allow the system to be used for both training and for actual emergency use. Each windlass rod 20 included in the system package will have a cavity 22 including an opening 24 to allow the insertion and removal of tourniquet(s) 16.

1. A device for a hemostasis system comprising a pressure band having two ends, a sliding buckle fixed at a first end and an unrestrained second end for engagement in the sliding buckle, a tourniquet with first and second ends, each tourniquet end attached to the pressure band, the device positioned to stop hemorrhage by application of a tourniquet portion of the pressure band on a hemorrhaging extremity with the pressure band ends attached; and a windlass rod having an elongate contoured cylindrical shape that extends along a longitudinal center axis, and includes a cavity for insertion and removal of the tourniquet.

2. The device for a hemostasis system as defined in claim 1, wherein said cavity is offset peripherally from a central, longitudinal axis of said windlass rod.

3. The device for a hemostasis system as defined in claim 2, wherein said windlass rod has a central portion with an essentially oval, elliptical cross section at said cavity.

4. The device for a hemostasis system as defined in claim 3, wherein an interior wall of said cavity is smooth.

5. The device for a hemostasis system as defined in claim 3, wherein an interior wall of the cavity includes a plurality of projections and depressions, within a range of 1.5 to 1.75 mm, measured from the surrounding flat, planar interior cavity wall to provide additional friction on the at least one tourniquet therein, when the tourniquet sliding buckle is fastened on a hemorrhaging extremity and pressure is applied by rotation of the windlass rod.

6, The device for a hemostasis system as defined in claims 4 or 5, including at least one flexible tab positioned over the cavity, which can be raised slightly to facilitate insertion of at least one tourniquet into the cavity, to protect an opening of the cavity.

7. The device for a hemostasis system as defined in claim 6, further comprising a plurality of indicators positioned adjacent to the cavity to ensure the at least one tourniquet is correctly fed into the cavity.

8. The device for a hemostasis system as defined in claim 7. wherein the windless rod is formed of a plastic material having longitudinal striated grooves positioned on an exterior surface of the windlass rod to enhance tactility of the windless rod.

9. The device for a hemostasis system as defined in claim 8, further comprising at lease one locking device removably positioned on the pressure band to hold the windlass rod in a desired retention position and allow its release from the retention position.

10. The device for a hemostasis system as defined in claim 9, wherein the locking device comprises a flexible “C” shaped clamp.

11. A hemostasis system to stop hemorrhages of the major extremities comprising a pressure band and an at least one tourniquet the ends of which are affixed to said pressure band, and wherein a windlass rod has an elongate contoured cylinder shape and extends along a central longitudinal axis, the windlass rod further comprising a cavity that is offset toward a windless rod surface, from the central longitudinal axis and into which is inserted an at least one tourniquet, and at least one flexible tab positioned on the cavity; further comprising indicator marks embossed in the device adjacent to the cavity to ensure the at least one tourniquet is correctly fed into the cavity; and wherein the windlass rod is formed of a plastic material.

12. The hemostasis system to stop hemorrhages of the major extremities as defined in claim 11, further comprising a sealed package of at least two windlass rods, each rod having a different color for identification of actual, practical use or for training, wherein the cavity of each windlass rod allows insertion and extraction of said at least one tourniquet.

13. The hemostasis system to stop hemorrhages of the major extremities as defined in claim 12, further comprising a smooth interior wall of the cavity.

14. The hemostasis system to stop hemorrhages of the major extremities as defined in claim 12, further comprising a friction means for retaining the at least one tourniquet in the cavity, said means positioned on an interior wall of the cavity.

15. The hemostasis system to stop hemorrhages of the major extremities as defined in claim 14, wherein the friction means comprises a gnarled portion on the interior walls of the cavity, the gnarled portion selected from a group comprising notches, scored, grooved, and rifling with spiral grooves, to provide additional friction on the at least one tourniquet therein.

16. The hemostasis system as defined in claim 15, further comprising longitudinal striated grooves on an external surface of the bar, wherein the grooves are parallel to the center axis and to each other.
